(19) United States
(12) Patent Application Publication
(10) Pub. No.: US 2011/0014269 A1
(43) Pub. Date: Jan. 20, 2011

(54) CIVAMIDE PATCH FOR LOCALIZED POST-INCISIONAL NEUROPATHIC PAIN

(75) Inventor: Joel E. Bernstein, Deerfield, IL (US)

Correspondence Address: BARNES & THORNBURG LLP, P.O. Box 2786, CHICAGO, IL, 60690-2786, US

(73) Assignee: WINSTON LABORATORIES, INC., Vernon Hills, IL (US)

(21) Appl. No.: 12/833,660

(22) Filed: Jul. 9, 2010

Related U.S. Application Data
(60) Provisional application No. 61/225,460, filed on Jul. 14, 2009.

(51) Int. Cl. A61K 9/70; A61K 31/165; A61P 29/00

(52) U.S. Cl. 424/447; 514/627

(57) ABSTRACT

A dermal method of treating chronic post-operative pain with a TRPV-1 receptor modulator. More specifically, a method for treating localized post-incisional neuropathic pain by administering a civamide-containing patch.

### BACKGROUND

This application claims priority from copending U.S. provisional application no. 61/225,460, filed Jul. 14, 2009, the content of which is herein incorporated by reference in its entirety.

Methods and compositions are disclosed for the treatment of localized post-incisional neuropathic pain by the dermal use of civamide. Surgical intervention often results in post-surgical pain, which over time can become independent of the condition that originally required surgery.

The pain remaining after healing of a surgical incision site itself is generally due to an ongoing inflammatory process, i.e. neuropathic pain, resulting from surgical trauma to peripheral nerves. This neuropathic pain accounts for a large proportion of pain experienced by post-surgical patients, the clinical significance of which is generally underestimated. The International Association for the Study of Pain has defined neuropathic pain as “pain due to a primary lesion or dysfunction of the peripheral or central nervous system”, experienced by a significant number of post-surgical patients. Neuropathic pain following surgery can occur in patients following breast surgery, in patients following thoracotomy, and in patients following coronary artery bypass surgery.

More specifically, post-mastectomy pain syndrome (PMPS) is a typical neuropathic pain, with associated symptoms (numbness, dysesthesia, edema and allodynia) located in the chest wall, axilla or arm, which persists beyond the typical three-month healing period following mastectomy. The same symptoms can also occur after lumpectomy, within the same timeframe. PMPS is typically caused by injury to nerves/tissues during the surgical procedure. Patients with PMPS present with pain along the distribution of the intercostal-brachial nerve, initiating at the location of the incision, and radiating on the same side of the body.

Chronic post thoracotomy pain syndrome (CPTPS) is defined as a pain that recurs or persists for at least two months following thoracotomy, occurring in more than 50% of cases. The surgical procedure can be extensive and associated with severe pain in itself, or in association with previous painful conditions such as COPD. Thoracotomy pain can also limit respiratory function, with negative effect on post-surgical recovery. CPTPS usually occurs as a result of surgical trauma or fibrosis of intercostal nerves. The pain is usually neuropathic, and in the distribution of the affected nerve.

Current methods for managing relief of post-incisional pain include intravenous formulations of opioids; oral formulations of opioids and NSAIDs (non-steroidal anti-inflammatory drugs); intranasal formulations of nicotine and opioids and transdermal formulations such as patches of nitroglycerin, clonidine, lidocaine or opioids.

Dermal therapies such as patches may be considered to be the least invasive of the approaches for alleviating post-incisional pain. Dermal drug delivery offers several advantages as it avoids the need for IV or intramuscular drug administration and is an option for patients unable to swallow oral medications. Dermal drug delivery also bypasses first-pass metabolism in the liver and overcomes concerns with drugs that are poorly absorbed in the gastrointestinal tract. However, the existing treatments for post-incisional pain can have undesirable side effects and unfavorable drug-drug interactions. In addition, for dermal delivery of opioids, other considerations such as the potential for abuse can make administration and management of treatment more difficult. Therefore there is still a need for an effective dermal delivered agent for relief of post-surgical pain.

One agent well-known for generalized typical relief of pain is capsaicin. The dosage forms of capsaicin that have been most widely studied clinically are homogeneous capsaicin-containing over-the-counter creams for dermal applications such as ZOSTRIX and AXSAIN. These products have been shown to be effective in managing painful conditions such as rheumatoid arthritis, osteoarthritis, diabetic neuropathy, post-herpetic neuralgia, post-mastectomy pain syndrome, cluster headaches and reflux sympathetic dystrophy.

Capsaicinoid gel formulations for relieving pre- and post-surgical pain have been disclosed in U.S. Patent Application Publication No. 20060148903; and injectable or implantable capsaicinoids are described for use in attenuating pain in U.S. Patent Application Publication No. 20050019436.

Furthermore, capsaicin and capsaicin analogues have been described for transdermal treatment of neuropathic pain in high concentration as disclosed in U.S. Pat. No. 6,239,180. Another transdermal formulation of capsaicin with pamabrom, a skin permeation enhancer and a non-steroidal anti-inflammatant, is disclosed in U.S. Pat. No. 5,665,378. Moreover, in U.S. Patent Application Publication No. 20060222690, a low concentration of capsaicin or capsaicin analogue (less than 1% by weight) in a patch for treating neuropathic pain is disclosed, but a penetration enhancer is required for effectiveness.

Watson et al. reported capsaicin topical cream to be effective in treatment of post-mastectomy pain syndrome in Pain, 51, (1992): 375-379.

However, chronic capsaicin treatment produces an insensitivity to almost all types of chemical irritation which persists for several months although responses to light touch and mechanical stimulation are unimpaired. Another disadvantage of treatment with capsaicin is that when it is administered in a patch formulation, pre-treatment with an anaesthetic and/or post-treatment cleaning of the skin may be required to render the therapy tolerable.

Therefore, there is still a need for effective pain-relieving compounds which do not cause unfavorable drug-drug interactions, undesirable side effects or discomfort for the patient.

### BRIEF SUMMARY OF THE DISCLOSURE

Methods and compositions for treating localized post-incisional neuropathic pain include administering an analgesically effective amount of cis-8-methyl-N-vanillyl-6-nonenamide or a pharmaceutically acceptable salt or prodrug thereof, dermally to a mammal in need thereof, wherein the pain can be, as examples, post-mastectomy and post-thoracotomy pain.

### DETAILED DESCRIPTION OF THE DISCLOSURE

Civamide (cis-8-methyl-N-vanillyl-6-nonenamide) is also known as zucapsaicin, It is a synthetic member of the capsaicinoid family and the synthetic cis-isomer of capsaicin. Civamide is a transient receptor potential cation channel, subfamily V, member 1 (TRPV-1) receptor modulator and has been found to be useful in the treatment of painful, inflammatory or allergic disorders, with significantly less of the burning and stinging associated with capsaicin's use. Civamide has been disclosed in U.S. Pat. No. 5,063,060, incorporated herein by reference. The compound is available from Sigma-Aldrich. U.S. Pat. No. 7,244,446 discloses topical or intranasal civamide for the diminishment of pain from arthritis, neuralgia or neuropathy; and U.S. Patent Application Publication No. 20050085552 discloses a single dose of intrathecally administered civamide for treating painful disorders including post-surgical pain.

Yet intrathecal injection can be disadvantageous because the procedure may lead to pain at the injection site as well as infection at the site or in the spinal column. Intrathecal injection is for systemic administration, which can be contrasted to the present methods of this disclosure which provide a topical or localized therapy.

Surprisingly, since civamide is efficacious at low concentration, it was found not necessary to use a skin permeation enhancer in the patch. The patch is thus effective without addition of a skin permeation enhancer.

Civamide can be used in the form of pharmaceutically acceptable salts derived from inorganic or organic acids. The phrase “pharmaceutically acceptable salt” means those salts which are, within the scope of sound medical judgment, suitable for use in contact with the tissues of mammals without undue toxicity, irritation, or allergic response.

Pharmaceutically acceptable salts are well-known in the art. For example, S. M. Berge et al. describe pharmaceutically acceptable salts in detail in J. Pharm. Sci. (1977), 66: 1 et seq. The salts can be prepared in situ during the final isolation and purification of the civamide or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsulfonate, digluconate, glycerolphospate, hemisulfate, heptanoate, hexanoate, fumarate, hydrochloride, hydrobromide, hydroiodide, 2-hydroxyethanesulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds described herein by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and non-toxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine and piperazine.

The term “pharmaceutically acceptable prodrug” or “prodrug” as used herein, represents those prodrugs of the compounds described herein which are, within the scope of sound medical judgment, suitable for use in contact with the tissues of mammals without undue toxicity, irritation, allergic response, and effective for their intended use. Prodrugs may be rapidly transformed in vivo to civamide for example by hydrolysis in blood.

Civamide formed by synthetic means or formed by in vivo biotransformation of a prodrug, is contemplated.

The compounds described herein can exist in unsolvated as well as solvated forms, including hydrated forms, such as hemi-hydrates. In general, the solvated forms, the pharmaceutically acceptable solvents such as water and ethanol among others are equivalent to the unsolvated forms for the purpose disclosed herein.

The term “dermal” as used herein means entry of the active agent to the skin from a patch.

The term “patch” as used herein means a medicated adhesive device which is placed on the skin to deliver a specific dose of medication to the skin.

A preferred vehicle for civamide dermal administration is a passive dermal system, wherein drug permeates directly to the skin. Dermal patch dosage forms preferably include a backing layer made of a pharmaceutically acceptable material which is impermeable to civamide. The backing layer preferably serves as a protective cover for the civamide, and may also provide a support function. Examples of materials suitable for making the backing layer are films of high and low density polyethylene, polypropylene, polyvinylchloride, polyurethane, polyesters such as poly(ethylene phthalate), metal foils, metal foil laminates of such suitable polymer films, and textile fabrics. The backing layer can be any appropriate thickness to provide the desired protective and support functions. A suitable thickness will be from around 10 to around 200 microns.

The adhesive layer preferably includes any adhesive known in the art that is pharmaceutically compatible with the dosage form and preferably hypoallergenic, such as polyacrylic adhesive polymers, acrylate copolymers and polyisobutylene adhesive polymers. In alternative embodiments of the invention, the adhesive is a hypoallergenic and pressure-sensitive contact adhesive. A preferred polymer is polyisobutylene.

It is understood that the civamide-containing patch can be co-administered with other active ingredients in various formulations including opioid analgesics, non-opioid analgesics, anti-depressants, compounds reacting with the N-methyl-D-aspartate (NMDA) receptor, compounds reacting with the nicotinic receptor and combinations thereof. The compounds are administered in formulations separate from the patch, and may be administered prior to, following or concurrently with administration of the civamide-containing patch.

Opioid analgesics may include, but are not limited to, codeine, morphine, hydromorphone, methadone, meperidine, levorphanol, fentanyl, butorphanol and pharmaceutically acceptable salts thereof. Non-opiod analgesics include acetylsalicyclic acid, acetaminophen, ibuprophen, gabapentin and pharmaceutically acceptable salts thereof.

Anti-depressants which may be co-administered include amitryptiline, desipramine, doxepin, imipramine, duloxetine and pharmaceutically acceptable salts thereof.

Compounds reacting with the NMDA receptor may include, but are not limited to, ketamine, norketamine and pharmaceutically acceptable salts thereof. Ketamine is a non-competitive NMDA receptor antagonist. Because ketamine is known to suppress breathing and circulatory function to a much lesser extent than most other available anesthetics, ketamine is often used as a first-line anesthetic for victims with unknown medical history, such as a victim of a car accident or other trauma. The compound which reacts with the nicotinic receptor may include, but is not limited to, nicotine, nornicotine and pharmaceutically acceptable salts thereof.

An analgesically effective amount of an analgesic agent means an amount of an agent capable of lowering the level of pain experienced by a patient. The level of pain experienced by a patient can be assessed by use of a visual analog scale (VAS) or a Likert-type scale. A VAS is a straight line with one end of the line representing no pain and the other end of the line representing the worst imaginable pain. Patients are asked to mark on the line where they considered their pain to be at each time point, and the length from no pain to the mark can be related to the length of the full scale. A Likert-type scale is a numeric rating scale, e.g. 0 to 5 or 0 to 10, based on degrees of agreement or disagreement to the statements at each end of the scale. Such pain scales can be applied to visualize an alteration of the level of pain a patient experiences during treatment, such as a reduction of the level of pain a patient or population of patients experiences before and after initiation of a pain therapy.

Therapeutic use of the patch can begin at any time subsequent when the normal healing process of a surgical incision should be complete, usually in the range of two to four months from the operation. If pain continues beyond the normal healing time, it is considered to be a chronic condition which can be addressed by the patch.

Moreover, the patch can be applied daily beginning one week prior to surgery to prevent occurrence of the chronic condition.

The patch may be worn daily for a defined duration. The patch may be worn by the patient for a period of from one to twenty-four hours. The patch can be used continuously for a period of weeks to a period of months. The patch can be discontinued for a defined period of time, and subsequently the patch can be applied again daily if required.

The term mammal includes human and animals.

The following Example is intended as an illustration of and not a limitation upon the scope of the invention as defined in the appended claims.

### Example 1

A patch manufactured by Aveva Drug Delivery Systems (Nitto Denko) is applied to the patient's trunk after a mastectomy or a lumpectomy, to address chronic and persisting pain which lasts long after the healing process has been completed; e.g. application beginning three months after surgery. The patch is applied directly over, or immediately adjacent to, the healed surgical site, to deliver medication locally. The patch contains 0.0075%-8-methyl-N-vanillyl-6-nonenamide (Sigma Aldrich), an adhesive material, a backing material and a release liner. In addition to the patch, the patient receives a narcotic and/or a non-steroidal medication.

After a single application of from one to twenty-four hours, the patch is removed. A new patch is applied for the same amount of time for the next six consecutive days, for a total of one week of treatment. After this initial course of treatment, efficacy is assessed by a verbal pain rating provided by the patient and a sedation score assessed by the physician. Incidences of nausea and vomiting are recorded and total additional narcotic and/or non-steroidal medication use is monitored. It is expected that a patient receiving the patch will show a marked decrease in total pain medication use. In addition, the patient's pain ratings should be lower as compared to a patient who received a placebo patch.

### Example 2

A patch manufactured by Aveva Drug Delivery Systems (Nitto Denko) is applied to the patient's trunk after thoracotomy, to address chronic and persisting pain which lasts long after the healing process has been completed; beginning two months after surgery. The patch is applied directly over, or immediately adjacent to, the healed surgical site, to deliver medication locally. The patch contains 0.0075%-8-methyl-N-vanillyl-6-nonenamide (Sigma Aldrich), an adhesive material, a backing material and a release liner. In addition to the patch, the patient receives a narcotic and/or a non-steroidal medication.

After a single application of from one to twenty-four hours, the patch is removed. A new patch is applied for the same amount of time for the next six consecutive days, for a total of one week of treatment. After this initial course of treatment, efficacy is assessed by a verbal pain rating provided by the patient and a sedation score assessed by the physician. Incidences of nausea and vomiting are recorded and total additional narcotic and/or non-steroidal medication use is monitored. It is expected that a patient receiving the patch show a marked decrease in total pain medication use. In addition, the patient's pain ratings should be lower as compared to a patient who received a placebo patch.

### Example 3

A patch manufactured by Aveva Drug Delivery Systems (Nitto Denko) is applied to the patient's remaining extremity after amputation of a limb, to address chronic and persisting phantom pain which lasts long after the healing process has been completed; beginning three months after surgery. The patch is applied directly over, or immediately adjacent to, the healed amputation site, to deliver medication locally. The patch contains 0.0075%-8-methyl-N-vanillyl-6-nonenamide (Sigma Aldrich), an adhesive material, a backing material and a release liner. In addition to the patch, the patient receives a narcotic and/or a non-steroidal medication.

After a single application of from one to twenty-four hours, the patch is removed. A new patch is applied for the same amount of time for the next six consecutive days, for a total of one week of treatment. After this initial course of treatment, efficacy is assessed by a verbal pain rating provided by the patient and a sedation score assessed by the physician. Incidences of nausea and vomiting are recorded and total additional narcotic and/or non-steroidal medication use is monitored. It is expected that a patient receiving the patch will show a marked decrease in total pain medication use. In addition, the patient's pain ratings should be lower as compared to a patient who received a placebo patch.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

I claim:

1. A method for treating localized post-incisional neuropathic pain comprising administering an analgesically effective amount of cis-8-methyl-N-vanillyl-6-nonenamide or a pharmaceutically acceptable salt or prodrug thereof dermally to a mammal in need thereof.

2. The method of claim 1 wherein said pain is post-mastectomy pain, post-lumpectomy pain or phantom limb pain.

3. The method of claim 1 wherein said pain is post-thoracotomy pain.

4. The method of claim 1 wherein said cis-8-methyl-N-vanillyl-6-nonenamide is in a patch having an adhesive material, a backing material, and a release liner.

5. The method of claim 4 wherein said cis-8-methyl-N-vanillyl-6-nonenamide is at a concentration of 0.001% to 0.1% by weight.

6. The method of claim 4 wherein said cis-8-methyl-N-vanillyl-6-nonenamide is at a concentration of 0.005% to 0.03% by weight.

7. The method of claim 4 wherein said patch is affixed to said mammal's skin subsequent to an operation for a defined duration of up to twenty-four hours.

8. The method of claim 4 wherein said patch is affixed to said mammal's skin prior to surgery for a defined duration of up to twenty-four hours.

9. The method of claim 8 wherein said patch is applied 24 hours prior to surgery.

10. The method of claim 1 wherein said mammal is a human.

11. The method of claim 2 wherein administration of said cis-8-methyl-N-vanillyl-6-nonenamide begins three months after mastectomy or lumpectomy.

12. The method of claim 3 wherein administration of said cis-8-methyl-N-vanillyl-6-nonenamide begins two months after thoracotomy.
